(19) United States
(12) Patent Application Publication
(10) Pub. No.: US 2007/0118130 A1
(43) Pub. Date: May 24, 2007

(54) Implant fixation methods and apparatus

(75) Inventors: Michael J. O'Neil, West Barnstable, MA (US); Michael A. Slivka, Taunton, MA (US); Ramon Alberto Ruberte, Ann Arbor, MI (US); Christine Rusbarsky, Quincy, MA (US); Scott Bruder, Sudbury, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/284,771

(22) Filed: Nov. 22, 2005

(51) Int. Cl. A61B 17/84; A61B 17/70; A61F 2/44

(52) U.S. Cl. 606/72; 623/17.11; 623/23.44; 606/61

(57) ABSTRACT

Various exemplary methods and devices are provided for fixing an implant to native tissue, such as bone. The devices can include a body with a tissue implant receiving opening and an aperture for receiving a fixation device. The body provides a large contact area for fixing the tissue implant to bone, such that the implant can be securely held in position without requiring penetration of the implant. Also provided is a tissue implant that includes pre-formed buckets for receiving fixation devices. The fixation devices can be positioned in the buckets of the tissue implant and implanted in bone holes without contacting native tissue.

### BACKGROUND OF THE INVENTION

Disease, advancing age, and trauma can lead to changes in various bones, discs, joints, and ligaments of the body. Some changes and trauma often manifest themselves in the form of damage or degeneration to a spinal disc. This condition often results in chronic back pain, which can be anywhere from mild to severe. This pain can sometimes be eliminated by removing the disc tissue between adjacent vertebral bodies and replacing it with a prosthetic device.

One type of procedure is spinal fusion, in which two adjacent vertebral bodies are jointed together after removing the intervening intervertebral disc. A prosthetic device is usually placed between the two adjacent vertebral bodies, in place of the removed disc, to fill the space left by the removed disc and to allow bone to grow between the two vertebral bodies. Alternatively, proposals have been made to replace the defective disc with an artificial disc that preserves the natural mobility between adjacent vertebral bodies. For example, such prostheses can include first and second plates for fixing to adjacent vertebral bodies, the plates having low friction contact surfaces that allow articulation.

As part of the surgical procedure to replace a disc, the soft tissue connecting adjacent vertebral bodies is at least partially cut-away. This can cause a loss of stability, particular where a mobility retaining prosthesis is utilized. To replace the function of the connective tissue, a tissue implant can be implanted. For example, a tissue implant can fixed at a first end to a first vertebral body and fixed at a second end to second vertebral body. To fix the tissue implants to bone, a surgeon can drive a screw or staple through the tissue implant and into the native tissue.

One drawback of such procedures is that the tissue implant can be weakened by the fixation procedure. When the screw or staple is driven through the implant it can create a weak spot, which may tear under load. For example, the screw or staple could be pulled through the tissue implant when tension is applied.

Accordingly, there remains a need for improved tissue implant constructs and devices for fixing tissue implants, particularly, methods and devices that can fix tissue while causing a minimum weakening of the implant.

### SUMMARY OF THE INVENTION

Described herein are methods and devices for fixing implants to bone. Unlike traditional fixation devices, such as bone screws or staples, the device described herein can include a large surface area for holding an implant in place. In addition, in at least one embodiment, the device is adapted to hold an implant without penetrating the implant. For example, an implant can be fixed in place between two surfaces of the device, between two devices, and/or between the device and a native tissue surface.

In one embodiment, the device includes a tissue fixation anchor having an elongate body with a longitudinal axis extending from a proximal end to a distal end. The body includes at least one opening for receiving a tissue implant and an aperture for receiving an expander. In one aspect, at least a portion of the aperture is coextensive with the tissue implant receiving opening and adapted to receive both a tissue implant and an expander. A tissue implant can be fixed within the implant receiving opening by positioning an expander within the aperture.

In one aspect, a tissue implant can be inserted into the tissue receiving opening and an expander can be inserted into the aperture such that the expander fixes tissue between an inner surface of the device and the expander. Alternatively, or additionally, the expander can radially expand the body of the device to fix tissue between an outer surface of the device and a native tissue surface.

The tissue receiving opening can extend through the body along an axis perpendicular to the longitudinal axis. For example, the opening can be defined by a transverse slot. In one aspect, the opening is positioned at the distal end of the body and extends to the distal end of the body. The aperture adapted to receive an expander can extend along the longitudinal axis of the body. In one aspect, the aperture can extend from the proximal end of the body to the tissue receiving opening.

In another embodiment described herein, a system for fixing a tissue implant to native tissue is provided. The system can include an anchor having an elongate body with a longitudinal axis extending from a proximal end to a distal end and an expander adapted for radial expansion. The body can include an opening for receiving a tissue implant and an aperture for receiving the expander. The body can also include an interior portion adapted to receive the expander and a tissue implant. In one aspect, the interior portion is positioned at the intersection of the opening and the aperture. When the expander is inserted into the device it can fix an implant positioned within the interior portion.

In yet another embodiment, a two-part tissue fixation device is provided. The device includes a first body having a generally wedge shape and at least one tissue implant receiving opening and a second body having a generally wedge shape with a proximal and distal end. The proximal end of the second body can include a slot adapted for receiving the first body. In one aspect, the first body is adapted to receive at least a portion of a tissue implant and the second body is adapted for receiving the first body, such that implanting the second body within native tissue fixes the tissue implant within the first body.

In yet another embodiment, A soft tissue implant is provided. The implant can include a longitudinally extending tissue implant having a body defined by a superior and inferior end, at least one of the superior and inferior ends including a recess adapted to receive an anchor. The implant body can be formed of a pliable, biocompatible material such that an anchor positioned within the at least one recess can be implanted in an aperture formed in a hard tissue surface without contacting the native tissue.

A method for fixing a tissue implant is also provided. In one embodiment, the method includes the steps of providing a pliable, biocompatible tissue implant having a longitudinally extending body and providing a tissue anchor. The body of the tissue implant can include a recess adapted to receiving a tissue anchor. The method further includes the step of positioning the tissue implant on a hard tissue surface having an aperture and implanting the tissue anchor through the tissue implant and into the aperture in the hard tissue surface. In one aspect, the anchor is implanted within the hard tissue surface without contacting native tissue.

Further features of the invention, its nature and various advantages, will be more apparent from the accompanying drawings and the following detailed description of the drawings and the preferred embodiments.

### BRIEF DESCRIPTION OF THE DRAWINGS

The invention can be more fully understood from the following detailed description taken in conjunction with the accompanying drawings, in which:

FIG. 1A is an anterior view of a vertebral column with a disc prosthesis positioned between adjacent vertebral bodies;

FIG. 1B is a side view of the vertebral column of FIG. 1A with one embodiment of a device described herein partially inserted into bone holes within the vertebral bodies;

FIG. 2A is a side view of another embodiment of a device described herein;

FIG. 2B is a perspective view of the device of FIG. 2A with a tissue implant positioned therein;

FIG. 3A is a front view of yet another embodiment of a device described herein;

FIG. 3B is a front view of another embodiment of the device illustrated in FIG. 3A;

FIG. 4A is a perspective view of another embodiment of a device described herein;

FIG. 4B is another perspective view of the device of FIG. 4A;

FIG. 4C is a side view of the device of FIG. 4A with an implant positioned therein;

FIG. 5 is a perspective view of another embodiment of a device described herein;

FIG. 6 is a perspective view of yet another embodiment of a device described herein;

FIG. 7A is a perspective view of still another embodiment of a device described herein;

FIG. 7B is a perspective view of the device of FIG. 7A with an implant disposed in a portion thereof;

FIG. 8A is a perspective view of another embodiment of the device of FIG. 7A;

FIG. 8B is a side view of the device of FIG. 8A;

FIG. 9 is a perspective view of another embodiment of the device of FIG. 7A;

FIG. 10 is a schematic of another embodiment of a device described herein;

FIG. 11A is a side view of a vertebral column and one embodiment of a device described herein; and

FIG. 11B is an anterior view of a vertebral column showing the device of FIG. 11A implanted thereon.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention

Various exemplary methods and devices are provided for fixing an implant to native tissue, such as bone. One such device includes a body extending along a longitudinal axis from a proximal end to a distal end, the body including at least one opening for receiving a tissue implant. The body provides a large contact area for fixing the tissue implant to bone, such that the implant can be securely held in position when the device is implanted. In one embodiment, the body can include at least one aperture for receiving a fixation device, such as an expander. A tissue implant can be positioned in the implant receiving opening and then fixed to bone by implanting an expander in the at least one aperture. For example, the tissue implant can be held between the expander and the body and/or between the body and a tissue surface. In an alternative embodiment, the tissue fixation device described herein includes two bodies that can be used together to fix a tissue implant in place. For example, a first body can include at least one implant receiving opening and the second body can include a slot for receiving the first body. A tissue implant can be fixed to native tissue by positioning the implant within the implant receiving slot of the first body, positioning the first body within the second body, and then implanting the first and second bodies. In yet another embodiment, a tissue implant is provided. The tissue implant can be adapted to receive a fixation device (e.g., a tissue anchor), such that the fixation device can be implanted through the implant and into an aperture within hard tissue. In one aspect, the fixation device can fix the implant in place in such a way that the fixation device does not contact native tissue.

Unlike conventional devices, such as bone screws, the apparatus described herein can fix a tissue implant to bone without penetrating the implant. Conventional tissue implant fixation procedures can have the potential to weaken a tissue implant, specifically in the region where the implant is penetrated. In addition, the devices described herein provide a large surface area, which can join the tissue implant to bone and provide additional securement. Thus, even if an implant is penetrated by a device of the type described herein, the implant contacting surfaces of the device can help to prevent tearing of the tissue implant when tension is applied to the implant.

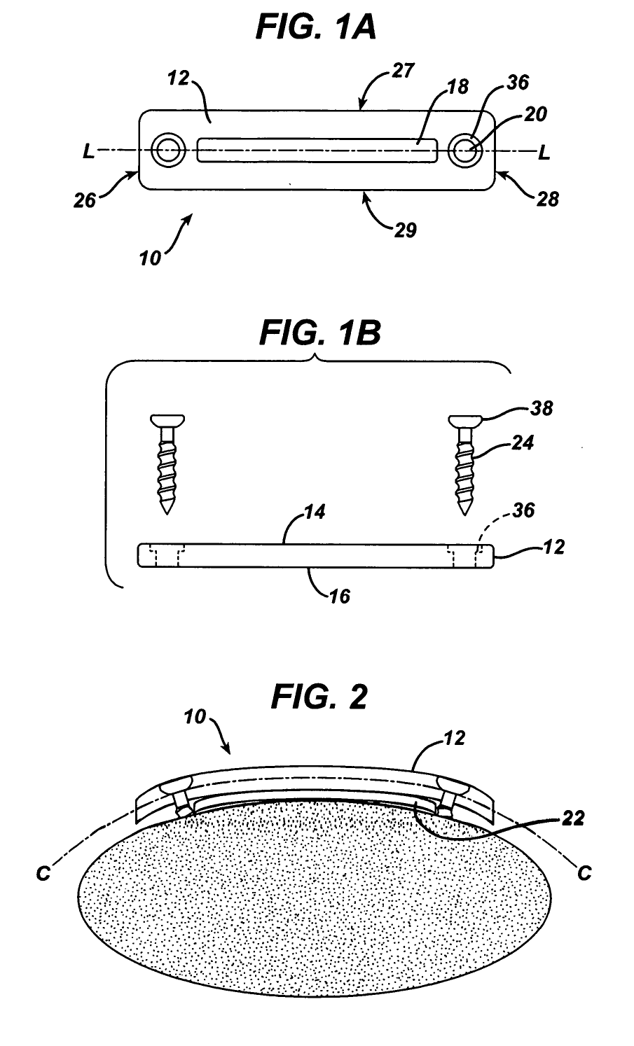

FIGS. 1A and 1B illustrate a vertebral column with superior vertebral body 2 and inferior vertebral body 4 and a disc prosthesis 6 positioned therebetween. To replace connective tissue cut away during implantation of disc prosthesis 6, a tissue implant 8 can be fixed to vertebral bodies 2, 4 with tissue implant fixation device 10. For example, vertebral bodies 2, 4 can include bone holes 12 adapted to receive device 10. Device 10 can mate with tissue implant 8 and be implanted in bone hole 12 to provide secure fixation of implant 8. While device 10 is described with respect to implantation on a vertebral body, one skilled in the art will appreciate that device 10 can be implanted on a variety of native tissue surfaces for fixing a variety of tissue implants. Exemplary tissue surfaces include the variety of hard tissue surfaces that may or may not be covered with soft tissue. In particular, the terms “hard tissue surface,” “bone,” and “vertebral body” do not exclude structures having a native soft tissue coating or layer that may, for example, include cartilage, tendons, ligaments, meniscus, or other soft tissue structures.

FIGS. 2A through 6 illustrate exemplary embodiments of device 10 including a body 14 having a generally elongate shape extending along a longitudinal axis from a proximal end 16 to a distal end 18. Body 14 can include an implant receiving opening 20 for receiving an implant and a aperture 22 for receiving a fixation device. In use, tissue implant 8 can be positioned in opening 20 and fixed in place by implanting device 10 into hole 12 in native tissue, such as, for example a vertebral body. Tissue implant 8 can be fixed between body 14 and expander 24 and/or between body 14 and a native tissue surface.

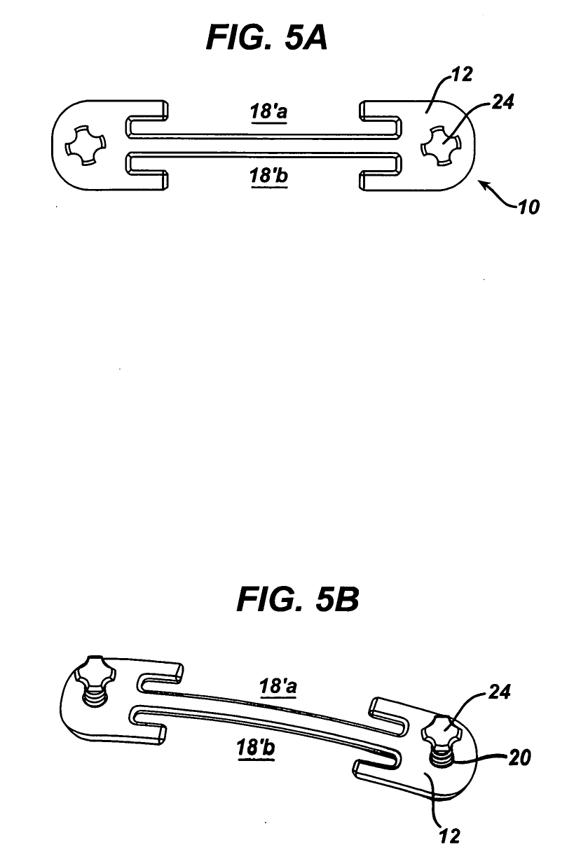

Body 14 can have a variety of shapes and sizes that are adapted for receipt within a bone hole. For example, body 14 can have a generally rectangular, cylindrical, triangular, or irregular shape. FIGS. 2A and 2B illustrate one embodiment of body 14 having a generally rectangular shape, while FIG. 5 illustrates a generally cylindrical body. To facilitate insertion into a bone hole, body 14 can include a rounded distal tip. Body 14 can be, in one embodiment, flexible or semi-rigid and/or deformable, such that device 10 will conform to the tissue in which it is implanted, thereby enhancing contact and securement between native tissue and the tissue implant. Alternatively, body 14 can be rigid and/or non-deformable. One skilled in the art will appreciate that body 14 can be produced from the variety of materials used in orthopaedic or implantable devices, such as, for example metals, polymers, ceramics, synthetics, and/or natural materials. Examples include Ti64, CoCr, resorbable and non-resorbable polymers, allografts, autografts, xenografts, and combinations thereof.

Opening 20 can be positioned within body 14 in a variety of locations. In one embodiment, opening 20 is positioned toward distal end 18 of body 14 as shown in FIG. 2A and 2B. For example, opening 20 can extend through the entire width of the distal portion of body 14 such that opening 20 defines a slot that extends through body 14 from a first side to a second side. In one aspect, opening 20 extends to the distal end of body 14 such that an implant can be inserted into device 10 from distal end 18.

Opening 20 can have a size and shaped adapted for receiving a variety of tissue implants. FIG. 2B illustrates body 14 with a generally planar implant 8 positioned within implant receiving opening 20. In one aspect opening 20 is sized such that it is larger than the thickness of an implant and the implant can be folded to accommodate the size of implant receiving opening. One skilled in the art will appreciate that a variety of alternatively shaped implants, such as implants having a circular cross-section, can be positioned in opening 20.

In one aspect, opening 20 extends through body 14 and defines first and second body segments 26, 28 (FIGS. 2A and 2B). Body 14 can be formed of flexible or malleable materials such that implant 8 forms a friction fit with body segments 26, 28. For example, implant 8 (or folded implant 8) can have a width that is slightly larger than the width W of opening 20. When implant is positioned in opening 20, body segments 26, 28 retain implant 8 in opening 20 with a friction fit.

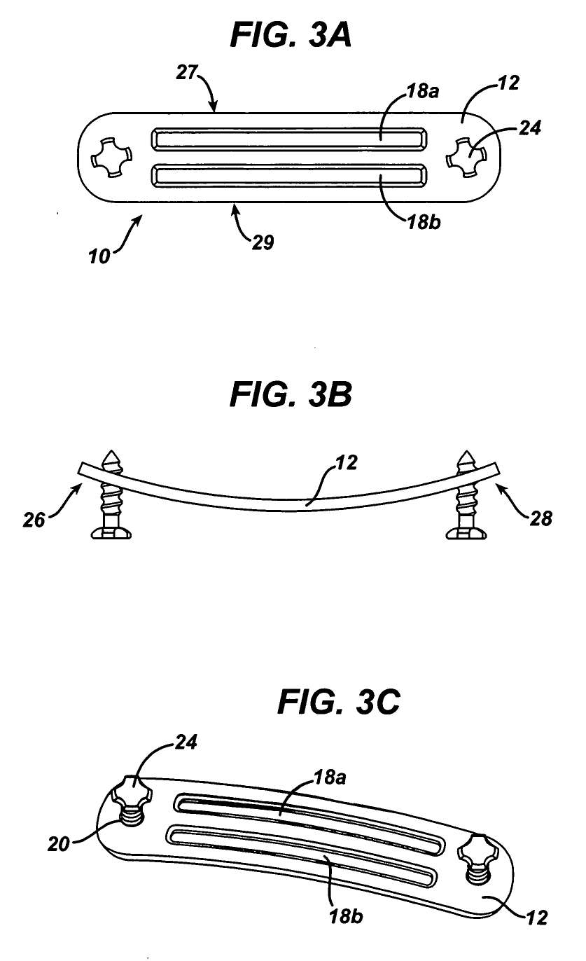

Opening 20 can have a variety of alternative configurations, including a “closed” configuration. FIGS. 3A and 3B illustrate device 10 with opening 20 extending through a central portion of body 14 between proximal and distal ends 16, 18. An implant can be threaded through opening 20 to position the implant in device 10. FIG. 3A illustrates body 14 having an opening 20 having a generally “V” shape and FIG. 3B illustrates opening 20 having a generally “U” shape. An implant, such as an implant having a planar shape, can be bent or folded such that it has a shape corresponding to opening 20. The implant can then be threaded through opening 20.

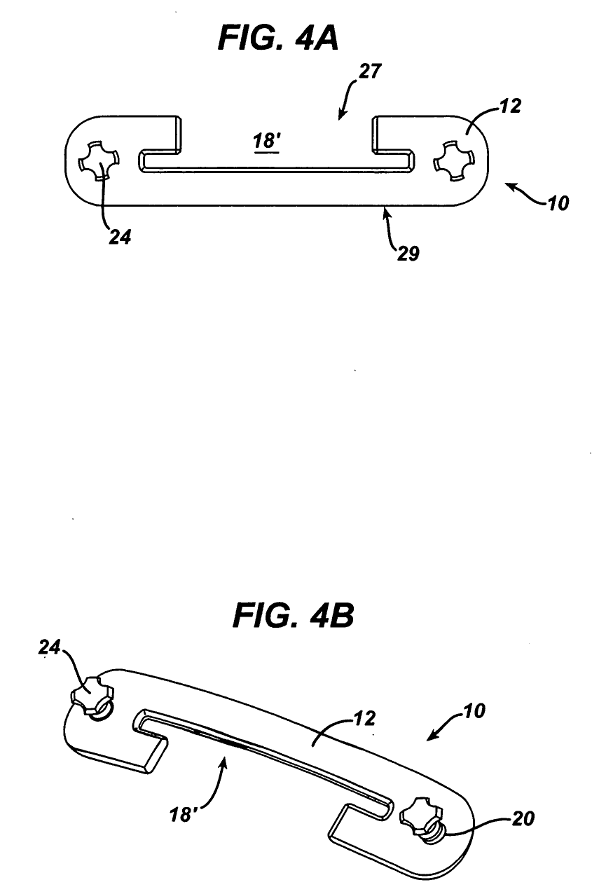

In another embodiment, opening 20 has a larger width and a body extension portion 30 positioned therein. An implant can be threaded through opening 20 and around the body extension portions 30. One such embodiment is illustrated in FIGS. 4A through 4C and includes two extension portions 30 that are defined by prongs that extend distally into opening 20. An implant can be threaded between one side of extension portions 30 and the wall of the opening. Alternatively, as shown in FIG. 4C, an implant can extended around the extension portions 30.

In one embodiment, opening 20 can be designed to retain an implant when the implant is positioned in opening 20. For example, an implant can be held in a friction fit as described above with respect to FIGS. 2A and 2B. Alternatively, an implant is loosely held in opening 20 and can be fixed in place with a fixation device (e.g., an expander) by pinning the implant between the fixation device and the inner wall of opening 20. For example, opening 20 can intersect aperture 22 such that an expander inserted into aperture 22 will contact an implant positioned within opening 20. The expander can be inserted into device 10 and expand body 14 to pin an implant between the expander and the wall of the opening 20. In yet another embodiment, an implant can be held in place by pinning the implant between the outer surface of body 14 and the wall of a bone hole when device 10 is implanted. For example, at least a portion the implant can be positioned between the outer surface of body 14 and the walls of bone hole 12 when device 10 is inserted into bone hole 12. Radially expanding body 14 with the expander will secure the implant between device 10 and the wall of the bone hole.

By fixing an implant between an expander and device 10 and/or between device 10 and native tissue, an implant can be fixed in place without penetrating the implant. In addition, the large contact areas between body 14 (i.e., the inner and/or outer surface of the device) and the implant provide greater securement than traditional devices (i.e., bone screws or staples).

A variety of fixation devices can be used with device 10 to fix device 10 within a bone hole. In one embodiment, the fixation device is an expander which increases the diameter of device 10 when it is inserted into aperture 22. For example, when device 10 is positioned within a bone hole, expander 24 can be inserted into aperture 22 to expand at least a portion of body 14 positioned within a bone hole. The expanded body 14 is thereby fixed in position, along with a tissue implant.

As shown in FIG. 5, body 14 can include an aperture 22 for receiving expander 24. In one aspect, aperture extends from the proximal portion of body 14 and extends along longitudinal axis L. Aperture 22 can intersect opening 20 such that they are coextensive in at least one portion of body 14. In the embodiment illustrated in FIGS. 2A and 2B, aperture 22 extends distally until it intersects opening 20 which is positioned transversely with respect to longitudinal axis L. When expander 24 is inserted into aperture 22, it can push body segments 26, 28 outward, fixing device 10 in a bone hole. Expander 24 can also fix an implant between an inner surface of body 14 and expander 24, such as for example between the inner wall of opening 20 and the expander.

In one embodiment, device 10 can include at least one proximal slot that allows body 14 to expand. In one embodiment, illustrated in FIG. 5, body 14 includes two proximal slots 34, 36 that define upper body segments 38, 40 and which intersect aperture 22. When expander 24 is positioned within aperture 22, the upper portion of body 14, particularly upper body segments 38, 40, are forced outward into contact with the walls of a bone hole. If driven far enough into aperture 22, expander 24 can also expand the distal portion of body 14, particularly lower body segments 26, 28.

As an alternative to slots, body 14 can be formed from a material which allows body 14 to expand when an expander is inserted into aperture 22. For example, body 14 can be formed from a pliable or flexible material that allows body 14 to increase in diameter when expander 24 is inserted therein.

As part of the procedure for fixing the implant within bone holes 12, the implant can be tensioned to provide improved tissue remodeling of the implant in the orientation of tensioning. For example, body 14, bone hole 12, and/or expander 24 can be can be configured such that expansion of device 10 and/or insertion of device 10 into bone hole 12 can be adjusted to tension the implant.

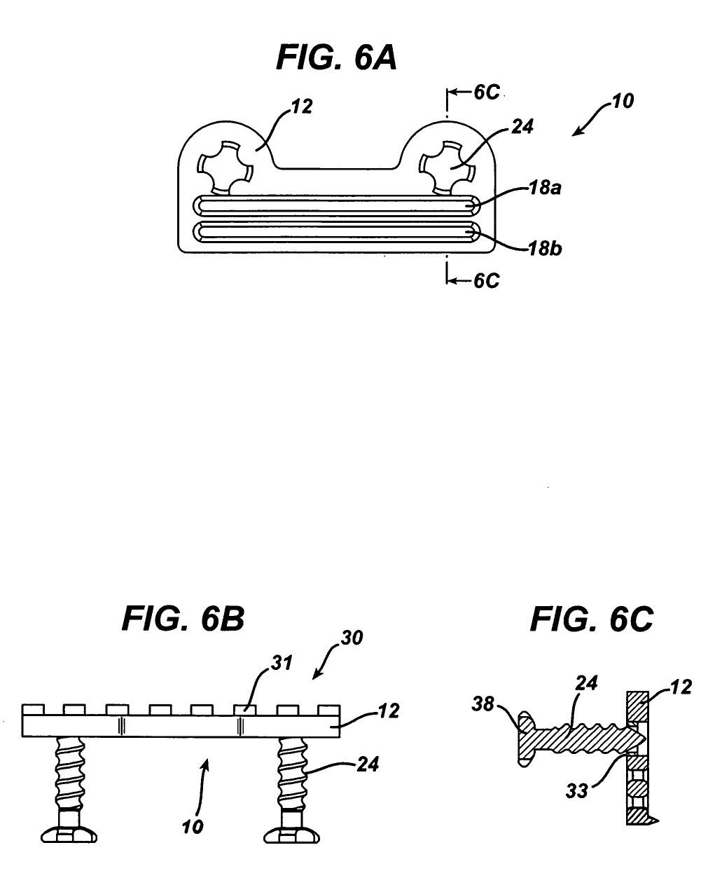

To assist with insertion of expander 24 into aperture 22, device 10 and/or expander 24 can include mating features 42. For example, the outer surface of expander 24 and the inner surface of aperture 22 can include threads, and the expander 24 can be screwed into aperture 22. In another exemplary embodiment, expander 24 can mate with aperture 22 in body 14 via a snap-fit. FIG. 6 illustrates device 10 with expander 24 positioned in aperture 22. Aperture 22 includes a proximal slot that can extend from the proximal end of body 14 into implant receiving opening 20. Expander 24 has a shape that corresponds to aperture 20 and opening 20. For example, the expander can have a “T” shaped cross-section with a planar proximal portion adapted to sit between segments 38, 40 and a distal portion adapted to mate with opening 20. Expander 24 can be positioned with the wider distal portion fitted within opening 20, such that proximal movement of the expander is prevented. The proximal portion of expander 24 can be positioned within aperture 22 such that upper body segments 38, 40 are expanded radially. In use, the device of FIG. 6 pins the implant between the outer surface of body 14 and the walls of a bone hole. One skilled in the art will appreciate that a variety of alternative expanders having a variety of mating features can be used to implant device 10 within a bone hole.

Body 14 can include a variety of features to assist with implantation/securement and/or tissue in-growth. In one aspect, outer surface 44 of body 14 includes surface features 46 that will contact native tissue when device 10 is implanted. Such surface features can include, for example, barbs, tines, fins, ribs, securement ridges, textured patterns, etchings, porous beading, and/or other coatings/treatments that promote soft or boney tissue apposition, integration, and/or resporbtion. In addition, or alternatively, surface features 46 can be adapted to resist movement out of a bone hole into which the device is implanted. For example, the surface features can be configured such that they will dig into bone if the device moves out of a bone hole. In one embodiment, shown in FIGS. 2A and 2B, transverse ridges 47 can encircle the outer surface 44 of body 14. The ridges have a distal-to-proximal slope and can include a bone penetrating proximal surface, such that the ridges do not resist implantation, but resist retropulsion.

In addition, surface features can be positioned on the inner surfaces of body 14 such that they contact an implant positioned within opening 20. For example, such surface features can be positioned on the inner surface of opening 20 and/or aperture 22.

In another embodiment described herein, device 100 is provided for fixing and implanting a tissue implant. In one aspect, device 100 includes a first body and a second body that work together to pin an implant therebetween and fix the implant to native tissue. The first body can be defined by a fixation device such as an expander that is adapted to receive at least a portion of a tissue implant and that is adapted to be inserted into the second body.

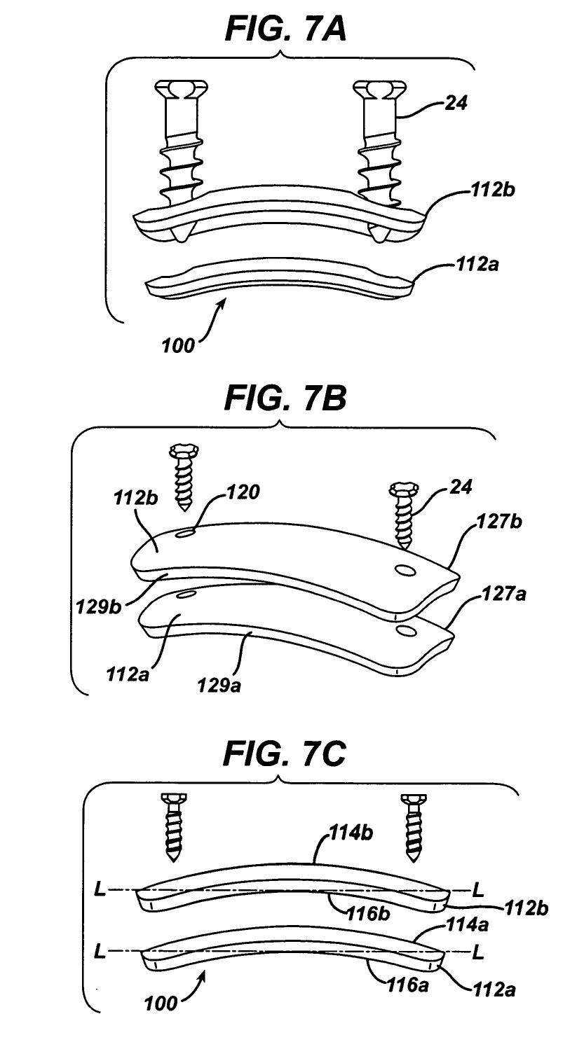

FIGS. 7A through 9 illustrate one exemplary embodiment of device 100 including first body 114a and second body 114b. First body 114a can include at least one tissue receiving opening 120 and has a size and shape such that when first body 114a is inserted into second body 114b, the second body is expanded. The second body 114b can include aperture 122 for receiving first body 114a. In one aspect, the aperture 122 of second body 114b is defined by a proximal slot that can be expanded when first body is inserted therein.

First and second bodies 114a, 114b can have a variety of shapes and sizes. In one embodiment, the first and second bodies are adapted for implantation in hard tissue, such as a vertebral body or other bone structure. First body 114a can have a wedge-type shape that extends from a proximal end 116a to a distal end 118a such that the thickness of body 114a decreases from the proximal to the distal direction. In one aspect body 114a is shaped and sized such that it can be received in aperture 122 of body 114b. As body 114a is forced into body 114b it causes the width of body 114b to expand, thereby fixing device 100 in place with hard tissue.

The proximal end of body 114a can have a variety of configurations, and in one embodiment illustrated in FIGS. 7A and 7B, the proximal end of body 114a can be defined by cover 152. Cover 152 can provide a surface for applying pressure to or for striking or impacting device 100 during the implantation procedure.

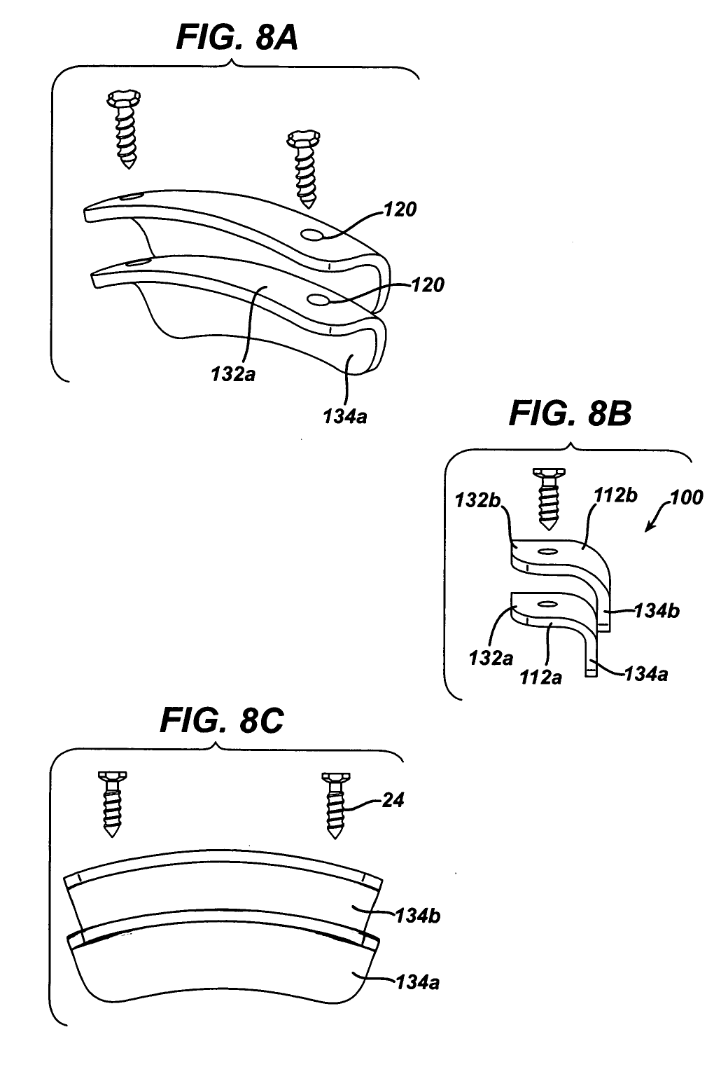

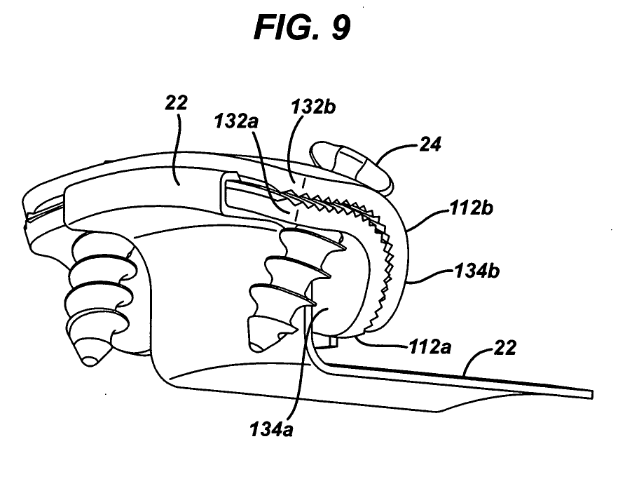

Between the proximal end 116a and distal end 118a, first body 114a can include any number of tissue receiving openings, including none, one, two, or more than two. In the embodiment illustrated in FIGS. 7A through 8B, first body 114a includes two openings 120a, 120b, through which an implant can be threaded. FIG. 7B illustrates body 114a with implant 8 threaded through opening 120a, through opening 120b, around the distal end 118a of body 114a, and then back through opening 120a. When first body 114a is inserted into second body 114b, implant 8 is held in place. In the embodiment illustrated in FIG. 9, body 114a has no implant receiving openings. However, an implant can be placed between body 114a and body 114b, and upon inserting body 114a into body 114b the implant will be fixed between the bodies when the device 100 is implanted.

Second body 114b can have a variety of shapes and sizes adapted for implanting in a hard tissue surface. In one embodiment, second body 114b also has a generally wedge-type shape with a proximal end 116b and a distal end 118b. Proximal end 116b can include aperture 122 for receiving body 114a. Distal end 118b can be defined by a bone penetrating point that facilitates driving device 100 into a hard tissue surface. One skilled in the art will appreciate that depending on the tissue into which device 100 will be implanted and the preparation provided (i.e., bone hole), second body 114b can have a variety of different shapes, such as, wedge, rectangular, oval, and irregular.

As mentioned above, devices 10 and 100 can be used to fix a variety of implants. In one embodiment, the implant is a soft tissue implant formed from materials, such as, for example resorbable and non-resorbable polymers, allografts, autografts, xenografts, and combinations thereof. In one aspect, the implant is formed from graft materials, such as, for example tendenous, cartilaginous, ligamentous, protein or collagen based materials, extra-cellular matrices (ECMs), or other synthetic resorbable or non-resorbable graft materials. In yet another embodiment, the implant is formed from small intestine submucosa (SIS). The implant can also be reinforced/enhanced with a variety of materials to augment its natural properties and/or promote tissue growth. In one aspect, the implant can include a coating or laminate of resorbable polymers and/or be treated or coated with a variety of growth factors, anti-coagulants and/or lubricants. In addition, or alternatively, the implant can be oriented to minimize local soft tissue adhesion by positioning the implant such that the treated or luminal side (if ECM) is oriented toward local soft tissue.

Fixation of the implant can also be augmented with materials to enhance securement, apposition, integration, and/or to fill voids created by a device/implant recessed within a bone hole. Exemplary augmentation materials can include adhesives (e.g., fibrin, polymeric glues, etc.), bone void fillers (e.g., hydroxyapatite, tricalcium phosphate, DBM putty, bone cement, and combinations thereof, etc.), injectable bone substitutes (e.g., collagen, BMP, etc.), growth factor delivery systems (e.g., osteoconductive matrix formulations (Healos), recombinant human growth/differentiation factor-5 (MP52), etc.), and combinations thereof. Augmentation can also be in the form of sutures wrapped around the implant and/or device. An alternative method of augmentation can include a plug or a second bone anchor that can fill voids between, above, or below the implanted device.

In another embodiment described herein, a tissue implant 200 is provided which is adapted to receive a fixation device therein. Instead of device, such as devices 10, 100 described above that receive an implant, the tissue implant includes buckets or recesses that are adapted to receive a fixation device. The fixation device is inserted into the bucket or recess of the implant to secure the implant within a bone hole. In one aspect, the fixation device can be implanted in the bone hole without penetrating the implant and without contacting the native tissue surfaces of the bone hole.

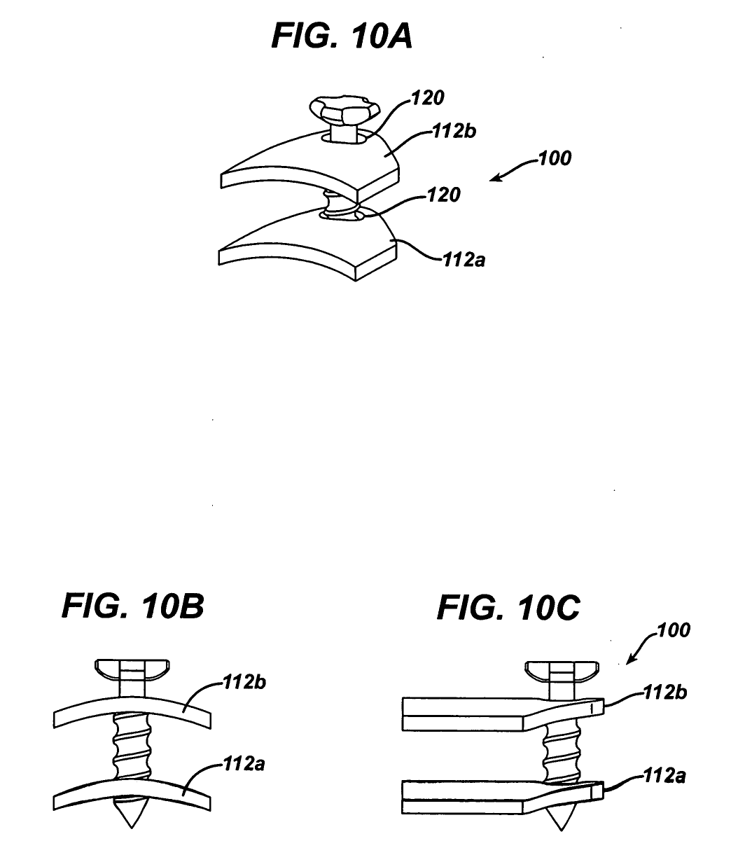

One embodiment of the tissue implant 200 is illustrated in FIG. 10, in which implant 200 includes an elongate body 214 that extends from an inferior end 216 to a superior end 218. Tissue implant 200 can include at least one recess 260 that is sized and shaped to receive a fixation device 224. In one embodiment, tissue implant 200 is adapted to receive a fixation device 224 that can be expanded radially. Alternatively, fixation device 224 can be implanted in bone hole 12 with a friction fit. One skilled in the art will appreciate that the variety of known fixation devices, including radially expanding tissue anchors, can be used with tissue implant 200.

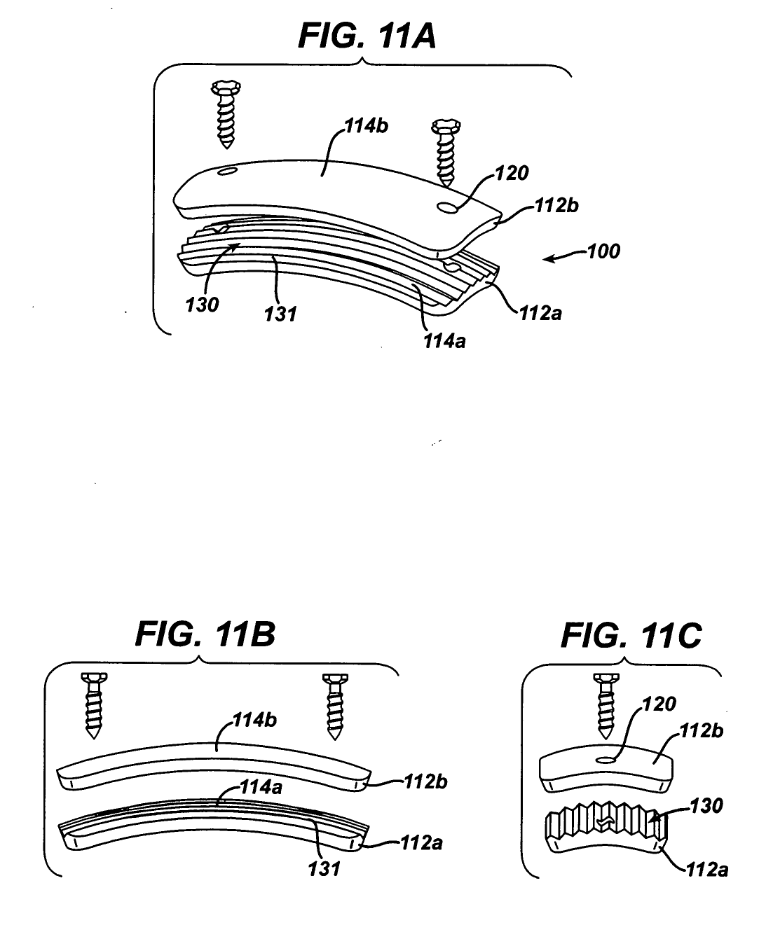

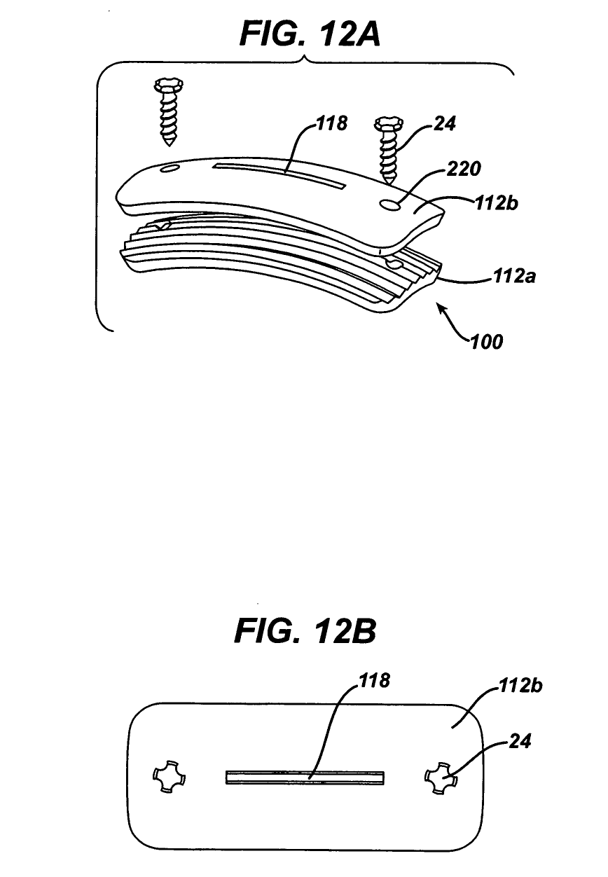

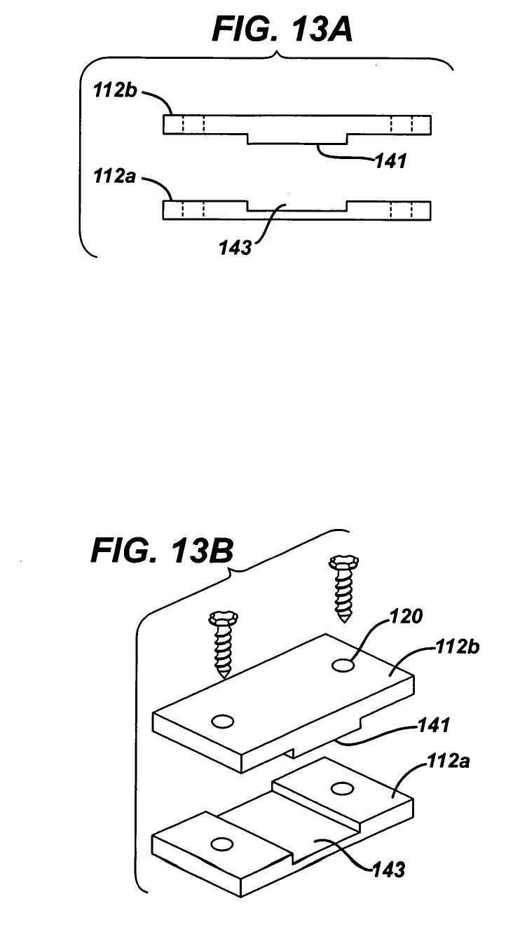

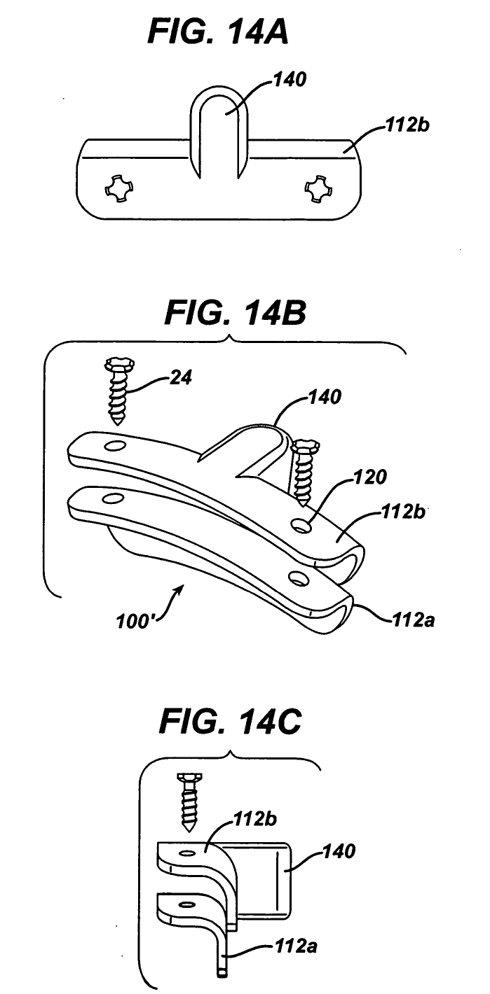

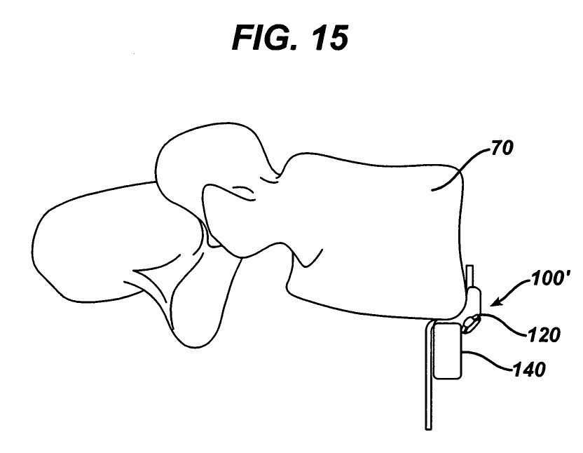

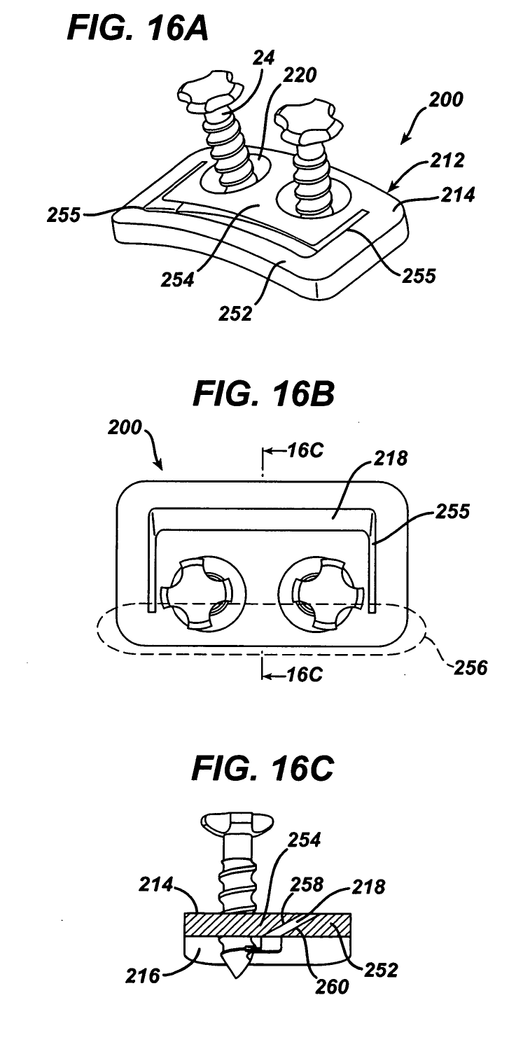

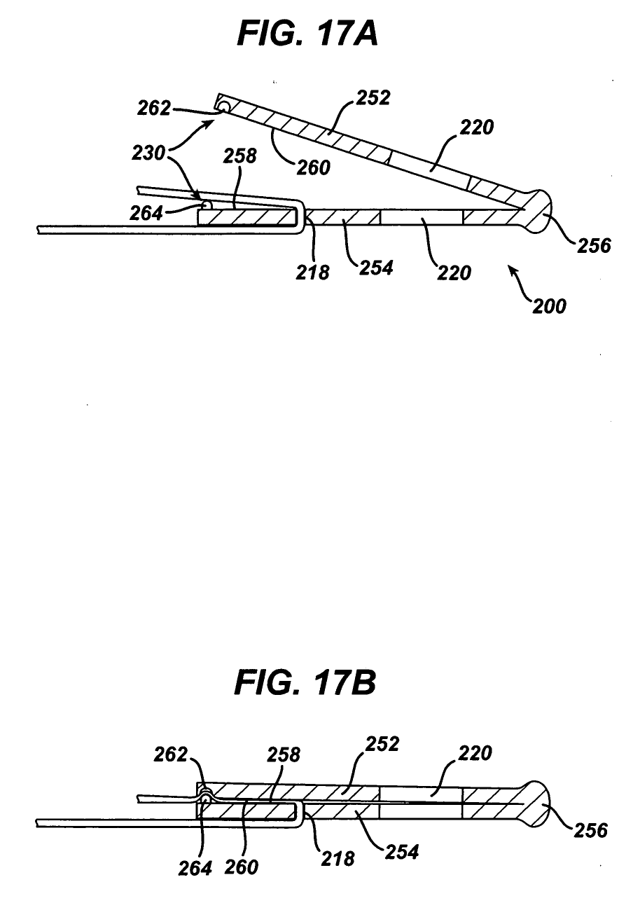

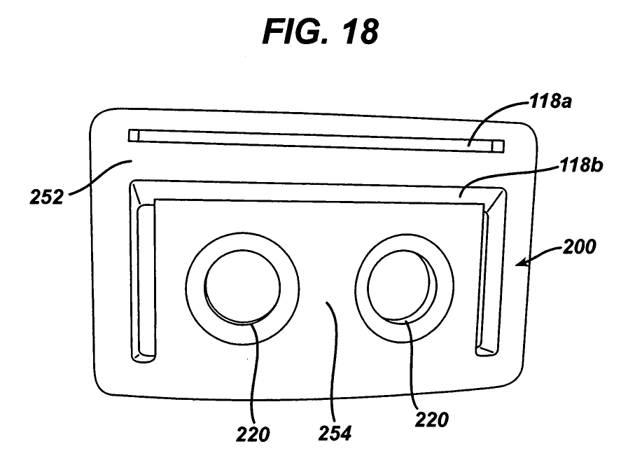

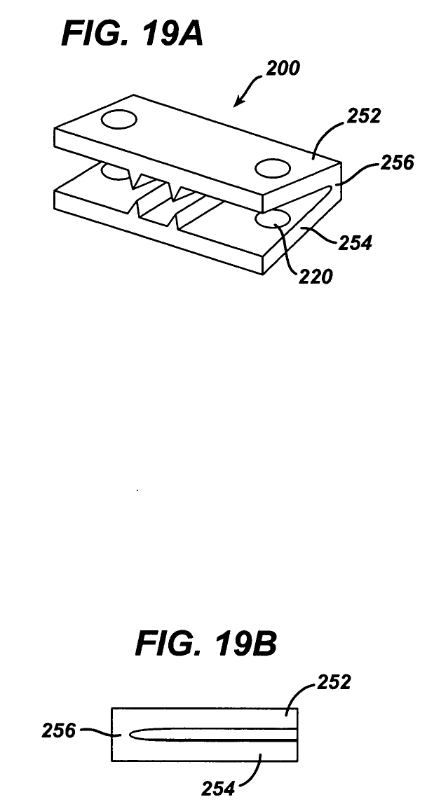

In one aspect, tissue implant 200 is sized and shaped for spanning adjacent vertebral bodies. The tissue implant 200 can include recesses 260 in the inferior end 216 and superior end 218 for receiving fixation devices. As shown in FIG. 10, the fixation device can be implanted into bone holes 12 on adjacent vertebral bodies with the tissue implant 200 positioned between the fixation devices and the inner surfaces of the bone holes. In another embodiment, illustrated in FIGS. 11A and 11B, device 200 can include multiple recesses 260 at inferior and superior ends 216, 218.

Body 214 can be formed of a biocompatible material that is in one embodiment flexible or pliable. When fixation devices 224 are implanted, they can be radially expanded such that they expand the recess 260 and fix device 200 in place. In an alternative embodiment, recesses 260 can be sized such that they are at least as wide as bone holes. Fixation devices 224 can be radially expanded without stretching body 214. In one embodiment, fixation devices are adapted to fix tissue implant 200 to bone without penetrating the tissue implant. For example, tissue implant 200 can be fixed between fixation device 224 and native tissue without fixation device 224 contacting the walls of bone hole 12.

Exemplary materials from which body 214 can be formed include metals, ceramics, polymers, synthetic and/or natural materials. Examples include, resorbable and non-resorbable polymers, allografts, autografts, xenografts, and combinations thereof. In one exemplary embodiment, body 214 is formed from tendenous, cartilaginous, ligamentous, protein or collagen based materials, extra-cellular matrices (ECMs), or other synthetic resorbable or non-resorbable graft materials. In yet another embodiment, body 214 is formed from small intestine submucosa (SIS). In addition, body 214 can include the various materials described above to enhance and/or augment implantation.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for fixing tissue implants, comprising:
an anchor having an elongate body with a longitudinal axis extending from a proximal end to a distal end, the body including an opening for receiving a tissue implant,
wherein a tissue implant can be fixed within the opening by implanting the body.

2. The device of claim 1, wherein the body includes a proximal aperture that is coextensive with the implant receiving opening.

3. The device of claim 2, wherein the aperture includes mating features adapted for mating with an expander.

4. The device of claim 2, wherein the tissue receiving opening is adapted to receive at least a portion of an expander that is inserted through the aperture, such that the expander can fix an implant positioned within the opening.

5. The device of claim 2, wherein the aperture extends along the longitudinal axis.

6. The device of claim 1, wherein the opening extends through the body along an axis perpendicular to the longitudinal axis.

7. The device of claim 1, wherein the opening is positioned at the distal end of the body.

8. The device of claim 1, wherein an outer surface of the body includes surface features adapted to mate with bone.

9. A system for fixing a tissue implant to native tissue, comprising:
An anchor having an elongate body with a longitudinal axis extending from a proximal end to a distal end, the body including an opening for receiving a tissue implant and an aperture for receiving an expander, the opening and the aperture intersecting within the body in a portion of the body adapted to receive an expander and a tissue implant; and
an expander adapted to mate with the aperture,
wherein the aperture is adapted such that positioning the expander within the aperture fixes a tissue implant positioned within the opening.

10. The system of claim 9, further comprising a tissue implant.

11. A soft tissue implant, comprising:
a longitudinally extending tissue implant having a body defined by a superior and inferior end, at least one of the superior and inferior ends including a recess adapted to receive an anchor, wherein the implant body is formed of a pliable, biocompatible material such that an anchor positioned within the at least one recess can be implanted in an aperture formed in a hard tissue surface without contacting native tissue.

12. The device of claim 11, wherein the implant is shaped and sized for implanting on a vertebral body.

13. The device of claim 11, wherein the body includes a first recess at the superior end and a second recess at the inferior end.

14. The device of claim 11, wherein the shape of the recess is complementary to the shape of an anchor.

15. A method for fixing a tissue implant, comprising:
providing a pliable, biocompatible tissue implant having a longitudinally extending body, the body including a recess adapted to receiving a tissue anchor;
providing a tissue anchor; and
positioning the recess within an aperture in a hard tissue surface and implanting the tissue anchor within the recess.

16. The method of claim 15, wherein the anchor is implanted within the aperture in the hard tissue surface without the anchor contacting native tissue.

17. The method of claim 15, wherein an outer side surface of the implanted anchor contacts an inner surface of the recess and fixes the tissue implant between the anchor and the aperture in the hard tissue surface.

18. A tissue fixation device, comprising:
a first body having a generally wedge shape and at least one tissue implant receiving opening; and
a second body having a generally wedge shape with a proximal and distal end, the proximal end including a slot adapted for receiving the first body,
wherein the first body is adapted to receive at least a portion of a tissue implant and the second body is adapted for receiving the first body, such that implanting the second body within native tissue fixes the tissue implant within the first body.

19. The device of claim 18, wherein the first body includes two tissue implant receiving openings.

20. The device of claim 18, wherein the second body is adapted for implanting in a hard tissue surface.
